(19) United States
(12) Patent Application Publication
(10) Pub. No.: US 2014/0050839 A1
(43) Pub. Date: Feb. 20, 2014

(54) METHOD OF EXTRACTING LECTIN FROM THE COMMON BEAN AS WELL AS A LECTIN PREPARATION

(71) Applicant: Pawel MICHALOWSKI, Michalow Grabina (PL)

(72) Inventor: Pawel MICHALOWSKI, Michalow Grabina (PL)

(21) Appl. No.: 13/763,877

(22) Filed: Feb. 11, 2013

Related U.S. Application Data
(63) Continuation-in-part of application No. PCT/PL2010/050048, filed on Aug. 12, 2010.

(51) Int. Cl. C07K 14/42; A23K 1/16; A23K 1/18

(52) U.S. Cl. CPC C07K 14/42; A23K 1/184; A23K 1/1631
USPC 426/656; 530/377

(57) ABSTRACT

A method of extracting lectin from the common bean as well as a lectin preparation. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/PL2010/050048, filed on Aug. 12, 2010. International Patent Application No. PCT/PL2010/050048 was pending as of the filing date of this application. The United States was an elected state in International Patent Application No. PCT/PL2010/050048.

### BACKGROUND

1. Technical Field

The subject of the present application is a method of producing a lectin preparation meant for use as feed additive for swine.

2. Background Information

Background information is for informational purposes only and does not necessarily admit that subsequently mentioned information and publications are prior art.

An invention known from the Polish application description P. 375476 “A method of producing a lectin preparation, a lectin preparation as well as a method of administering a lectin preparation for mammals” assumed the use of preservatives in order to protect the product in the form of a powered, did not indicate the need or desire for use of a preservative during extraction and extract condensation, and for example during the storage period of the extract as well as the concentrated extract in order to perform necessary and/or desired activity analyses as well as during the intervening period before the initiation of further stages of production. In certain situations, the known and abovementioned description No. P.375476 allowed the apportionment of the extract of a defined biological activity directly into dosing containers, bypassing the drying stage. This, however, required and/or desired standard methods of introducing a preservative prior to apportionment, and did not affect either the extraction method nor its results. Other lectin production methods known from literature made use of multistage extraction procedures, pH changes of the extract and multiple filtration, salt precipitation, dialysis and lyophilisation as the basis of a process for the production of a solid product. The abovementioned method facilitates the production of lectin preparations of a predefined activity limited by the presence of ballast substances in the extraction raw materials. There is thus still a need or desire to deliver such a method of extracting that would make it possible to produce a product with an increased activity. The goal of the present application is to deliver a method of producing highly active finished product in powder form from bean seeds for use as a feed additive for swine. Unexpectedly, the abovementioned problem was solved by the present application.

### OBJECT OR OBJECTS

An object of the present application is to provide a method of the industrial production of a lectin preparation from bean seeds based on that the bean seeds are broken up, the bean pulp is extracted in an acidic environment in the presence of antioxidants and a preservative, wherein the extraction is performed at a lowered temperature, in one possible embodiment from 4° C. to 8° C., wherein in the initial phase of the extraction are the phases mixed and then the extract is concentrated, in one possible embodiment on membranes, and a poorly volatile preservative is added to the thusly produced PHA fraction and it is then dried, in one possible embodiment by spraying, resulting in a lectin preparation with an active lectin content no lower than 128 HU/100 mg of preparation. In at least one possible embodiment of a method according to the present application, the extraction is performed using an extraction system comprising acetic acid, an antioxidant, as well as a highly volatile short-chained organic acid or its salt at a lowered temperature. In another possible embodiment of a method according to the present application, the solid and liquid phases of the extract are mixed during the first stage of extracting, in one possible embodiment within 60 minutes. In at least one possible embodiment of a method according to the present application, the second part of the extraction process occurs without phase mixing, in one possible embodiment within 7 hours.

The second subject the present application is a feed additive for swine produced from seeds of the common bean, characterized in that the lectin activity is no less than 128 HU/100 mg of preparation with a decreased level of ballast substances. In one possible embodiment, a feed additive according to the present application is obtained using a method according to the present application.

### SUMMARY

Unexpectedly, it turned out that a great reduction in the mixing time during the extraction, change in temperature range (and thus a change in the dynamics and characteristics of this process) greatly improves the efficiency and selectivity of PHA extraction (Latin: Phytohaemagglutinin), which in effect facilitates and essentially guarantees or promotes the production of a product with a higher biological activity and lower level of ballasting substances in relation to methods of producing PHA lectins known from and described in literature. The nature of the new method of producing a lectin preparation in powder form is to create a novel extraction system, wherein the changes introduced into the extraction system, omission of mixing and the parallel decrease or substantially parallel decrease in process temperature, significantly influence the selectivity of the extracting process, expressed in terms of a decreased ballast substance content in the extract, with an unchanged lectin level, and thus an unchanged activity level both of the extract and the lectin preparation made of it. The present application solves the problem of the efficiency of extracting PHA from legume seeds, here the common bean, and essentially ensures and/or promotes a decreased level of ballast substances in the individual phases of the production of the lectin preparation. The novel method of producing a lectin preparation may possibly relate to the extraction phase of forms of lectin from bean seeds, and facilitates the production of highly active lectin preparations in powder form. As a consequence, the activity of the final powdered product can be increased from 128 HU/100 mg to 256 HU/100 mg, and, by the same token, significantly lower the cost of production per 1000 HU. The described method leads to the production of a novel, different lectin preparation with an increased mass content of five forms of lectins in relation to the remaining organic substances comprised and isolated with the extract using the methods of the invention in patent application P. 375476 entitled “A method of producing a lectin preparation, a lectin preparation as well as a method of administering the lectin preparation to mammals”, as well as the invention under application number FL 384706 “A lectin feed additive as well as an industrial method of producing it”. Both the product and intermediate phases are controlled through the determination of hemagglutinin activity (HU), defined as the amount of material expressed in mg/ml in the final dilution causing 50% agglutination of a 2% erythrocyte suspension at a temperature of +25° C according to the method of Pusztai and Watt, 1974, with a modification. Another method of indirect control of the extraction and concentration processes, for example for technical-technological purposes, is to determine dry mass. During work on the improvement of the effectiveness of the production of lectin preparations as well as technologies for variable conditions of extraction, it unexpectedly turned out that the use of technological regimes of extraction, alternate to extant ones, not only improves the conditions of PHA extraction from leguminous plant seed masses, here bean seeds, which is evidenced by the increased hemagglutinin activity of the extract and the powder made from it during subsequent processes in relation to dry mass determinations, thus confirming the decrease of ballast substance content in the extract and product, and facilitates the production of a lectin product with an activity greater than 128 HU/100 mg. The possibility of producing the biological activity of the powder over 128 HU/100 mg not only decreases the costs of producing the same amount of powder, but also facilitates the design of novel lectin product formulations.

### EXAMPLE 1

Extraction of PHA from bean seeds and production of the final product using the method according to the present application.

### Preparation of Common Bean Seeds.

Seeds of the common bean were milled in a mill with internal cooling and milling temperature control. The milling process temperature varied from +34° C. to 40° C. The milled particle diameter was set to grains with a diameter of 1 mm, which in practice yields 98% of a fraction with grain diameters of 0.98-1.02 mm.

### Preparation of the Extraction Solution:

An acetic acid solution is prepared for the extraction using a slow agitator, where glacial acetic acid is mixed into water at a rate of 1.1 g acetic acid in 1000 ml solution. This solution is supplemented with 0.1 g ascorbic acid per every liter of solution well as propionic acid at 0.1 g per liter of solution. The amount of extraction solution is made dependent on the mass of milled bean seeds. The recommended ratio is 10:1.

### Extraction Process:

For the extraction, a measured amount of ground common bean seeds are added to the extraction solution and mixing is initiated. After 10 minutes, the pH control of the extraction process begins. The recommended pH for the extraction process is in the range 5.0±0.07. Any possible pH corrections should be made after a minimum 30 min of extracting, because after that point one can start to speak of the pH stabilization of the extract system. Should a pH correction be needed and/or desired, after 20 minutes the correction result should be checked. The extraction temperature should be +4° C. −+8° C. The mixing should last 1 hour altogether, whereafter the mixture is set aside without mixing for another 7 hours.

### Centrifugation of the Extract:

The separation of the solids occurs on high-RPM drum centrifuges designed for the separation of protein precipitates. Drum diameter is 200 mm, and rotational speed is 18 000 RPM. The process is periodic, and the cycle is determined by drum volume.

The separated post-extraction residue, and the protein-rich product, are processed separately in order to prepare it for use as an animal feed.

### Further Extract Processing:

The clarified extract is concentrated using reverse osmosis. The initial parameters of the extract are: dry mass 1.242 %, activity 64 HU/100 ml. The concentrated solution is analyzed for hemagglutinin activity and dry mass content. Immediately or substantially immediately prior to the spray drying, the concentrated extract is supplemented with an appropriate, calculated amount of maltodextrin and alternatively sodium N-propyl p-hydroxybenzoate or sodium N-ethyl p-hydroxybenzoate. The spray drying process is performed at an inlet temperature not higher than +180° C. The product is analyzed for biological activity, and a portion of material is formulated, which is analyzed according to EU requirements for feed additives. The resulting product is a pale (white to cream-colored) powder, highly pourable, a tendency to clump and easily forming a suspension in water. The powder is fully compatible with commonly used mechanical pouring machines. The hemagglutinin activity is no lesser than 128 HU/100 mg,; Hg, As, Pb, and Cd content is much lower than EU norms; microbiological purity is compliant, and has a shelf-life of 3 years from production.

### EXAMPLE 2

Hemagglutination test for determining the activity of the preparation

### Preparation of Erythrocytes

1 ml of blood collected from a Wistar rat (with 1 drop of heparin) was diluted 1:20 in physiological saline (0.9% NaCl) and centrifuged (2000 RPM, 15 mm, 4° C.). The supernatant was poured off along with the interphase. The erythrocytes were resuspended in physiological saline and washed as shown above 3 times. Next, a 2% erythrocyte solution was made (1:50 dilution).

### Preparation of the Preparation

For the analysis, 200 mg of the powdered preparation were taken and dissolved in 8 ml of physiological saline. Final concentration −25 mg/ml.

A series of twofold dilutions was prepared: Dilutions: 1× 2× 4× 8× 16× 32× 64× 128× 256×

### Test

The test were performed simultaneously or substantially simultaneously in Eppendorf tubes as well as on microscope slides (not degreased!).

The reaction mixture in the Eppendorf tubes comprises: 100 μl physiological saline, 100 μl erythrocytes 2% and 100 μl of the examined solution (appropriate solution). After thoroughly mixing the components, 50 μl is collected from each examined sample and put onto the slides and incubated at room temperature for about 30 minutes to an hour (care should be taken that the drops do not dry out). The remaining part of the mixture is incubated in closed Eppendorfs (no less than 1 hour, elongation does not affect the readout). After the incubation, the contents of the Eppendorf tubes are mixed with a pipette to free the precipitate off the walls, and then 50 μl are collected and placed on a microscope slide. The test is performed in the presence of a negative control: 200 μl physiological saline and 100 μl of 2% erythrocytes. The test result is read out under a microscope at 250× magnification. The results are written using the marks: +, +/−, −, where ++ means the presence of hemagglutinated erythrocytes, and − is their absence. To simplify, one can omit the incubation on microscope slides, as sufficient results are obtained in the incubation in closed Eppendorf tubes.

### Determination of the Hemagglutinin Units in the Preparation

One hemagglutinin unit (1 HU) is 1 mg of lectin preparation, whose concentration of 1 mg/ml causes the hemagglutination of at least 50% of erythrocytes in accordance with the abovementioned procedure of determining the degree of hemagglutination. The activity of the above preparation is 1 HU/mg.

The number of hemagglutination units (HU) in 1 mg of the examined lectin preparation is denoted as the inverse of the concentration of the solution (shown in mg/ml) in the next dilution, wherein one still observes the hemagglutination of at least 50% of erythrocytes.

For a preparation according to the present application, activity expressed in hemagglutination units is usually given calculated per 100 mg of preparation.

### EXAMPLE 3

PHA extraction from bean seeds and production of the ready product using the method according to the invention of patent PL 384706

### Preparation of Common Bean Seeds

Seeds of the common bean were milled in a mill with internal cooling and milling temperature control. The milling process temperature varied from +34° C. to 40° C. The milled particle diameter was set to grains with a diameter of 1 mm, which in practice yields 98% of a fraction with grain diameters of 0.98-1.02 mm.

### Preparation of the Extraction Solution:

An acetic acid solution is prepared for the extraction using a slow agitator, where glacial acetic acid is mixed into water at a rate of 1.1 g acetic acid in 1000 ml solution. This solution is supplemented with 0.1 g ascorbic acid per every liter of solution well as propionic acid at 0.1 g per liter of solution. The amount of extraction solution is made dependent on the mass of milled bean seeds. The recommended ratio is 10:1.

For the extraction, a measured amount of ground common bean seeds are added to the extraction solution and mixing is initiated. After 10 minutes, the pH control of the extraction process begins. The recommended pH for the extraction process is in the range 5.0±0.07. Any possible pH corrections should be made after a minimum 30 min of extracting, because after that point one can start to speak of the pH stabilization of the extract system. The extraction process does not require and/or desire exact and/or approximate temperature regimes because it is not significantly exergonic. The extraction temperature should be +16° C.-+25° C. The mixing should last 4 hours altogether, whereafter the mixture is set aside without mixing for another 4 hours.

### Centrifugation of the Extract:

The separation of the solids occurs on high-speed drum centrifuges designed for the separation of protein precipitates. Drum diameter is 200 mm, and rotational speed is 18 000 RPM. The process is periodic, and the cycle is determined by drum volume. The separated post-extraction residue, and the protein-rich product, are processed separately in order to prepare it for use as an animal feed.

### Further Extract Processing:

The clarified extract is concentrated using reverse osmosis. The initial parameters of the extract are: dry mass 1.242 %, activity 64 HU/100 ml. The concentrated solution is analyzed for hemagglutinin activity and dry mass content. Immediately or substantially immediately prior to the spray drying, the concentrated extract is supplemented with an appropriate, calculated amount of maltodextrin and alternatively sodium N-propyl p-hydroxybenzoate or sodium N-ethyl p-hydroxybenzoate. The spray drying process is performed at an inlet temperature not higher than +180° C. The product is analyzed for biological activity, and a portion of material is formulated, which is analyzed according to EU requirements for feed additives. The resulting product is a pale (white to cream-colored) powder, highly pourable, a tendency to clump and easily forming a suspension in water. The powder is fully compatible with commonly used mechanical pouring machines. The product was used in tests on two Polish swine farms. Powder activity was 128 HU/100 mg.

The extraction results according to the new and old methods as well as the activity of the product resulting from them, lectin powder for preparing a suspension ex tempore is shown in Table 1 below.

TABLE 1
PHA activity of the extract and final product according to
prior art and the present application

| | Extraction according | | | Extraction according | | |
|---|---|---|---|---|---|---|
| | the novel method | | | to invention FL | | |
| | (Example 1) | | | 384706 (Example 2) | | |
| | Sample number | | | | | |
| | 1 | 2 | 3 | 4 | 5 | 6 |
| Dry mass | 1,242 | 1,242 | 1,241 | 1,286 | 1,282 | 1,279 |
| content (%) | | | | | | |
| PHA activity | 64 | 64 | 64 | 64 | 64 | 64 |
| of the | | | | | | |
| extract | | | | | | |
| HU/100 ml | | | | | | |
| Final product | 256 | 256 | 256 | 128 | 128 | 128 |
| activity | | | | | | |
| HU/100 mg | | | | | | |

A method of the industrial production of a lectin preparation from bean seeds, in which a lectin fraction is extracted from bean seeds in an extraction system comprising acetic acid, an antioxidant, as well as a highly volatile organic acid or its salt, at a lowered temperature and substantially without mixing, and subsequently, after concentrating, in one possible embodiment on membranes, the resulting PHA fraction is supplemented with a poorly volatile preservative and it is dried by spray-drying resulting in a lectin preparation with a lectin activity of no less than 128 HU/100 mg of preparation, with a lowered level of ballasting substances.

One feature or aspect of an embodiment is believed at the time of the filing of this patent application to possibly reside broadly in an industrial method of producing a lectin preparation from bean seeds where the bean seeds are milled, the bean mass is extracted in an acidic environment in the presence of antioxidants and a preservative, wherein the extraction is performed at a lowered temperature, in one possible embodiment from 4° C. to 8° C., wherein phase mixing is performed during the initial stage of extraction and then the extract is concentrated, in one possible embodiment on membranes, and a thusly produced PHA fraction is supplemented with a poorly volatile preservative and is dried, in one possible embodiment by spraying, resulting in a lectin preparation comprising a lectin activity of no less than 128 HU/100 mg preparation.

Another feature or aspect of an embodiment is believed at the time of the filing of this patent application to possibly reside broadly in the method, wherein the extraction is performed using an extraction system comprising acetic acid, an antioxidant, as well as a highly volatile, short-chained organic acid or its salt at a lowered temperature.

Yet another feature or aspect of an embodiment is believed at the time of the filing of this patent application to possibly reside broadly in the method, wherein the solid and liquid phases are mixed in the extract during the first stage of extracting, in one possible embodiment within 60 minutes.

Still another feature or aspect of an embodiment is believed at the time of the filing of this patent application to possibly reside broadly in the method, wherein the second stage of the extraction process occurs without phase mixing, in one possible embodiment within 7 hours.

A further feature or aspect of an embodiment is believed at the time of the filing of this patent application to possibly reside broadly in a feed additive for swine obtained from seeds of the common bean, wherein it comprises lectins with an activity of no less than 128 HU/100 mg of preparation with a lowered level of ballasting substances.

Another feature or aspect of an embodiment is believed at the time of the filing of this patent application to possibly reside broadly in the feed additive, wherein it was obtained using the method according to the present application.

The components disclosed in the patents, patent applications, patent publications, and other documents disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications, patent publications, and other documents cited herein, and in the Declaration attached hereto, are hereby incorporated by reference as if set forth in their entirety herein except for the exceptions indicated herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, patent applications, patent publications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, patent applications, patent publications, and other documents either incorporated by reference or not incorporated by reference.

All of the patents, patent applications, patent publications, and other documents, except for the exceptions indicated herein, which were cited in the International Search Report dated Feb. 22, 2012, and/or cited elsewhere, as well as the International Search Report document itself, are hereby incorporated by reference as if set forth in their entirety herein except for the exceptions indicated herein, as follows: EP 1 734 131, having the title “A process of manufacturing a lectin preparation, the lectin preparation and methods of administration of the preparation to mammals,” published on Dec. 20, 2006; WO 97/49420, having the title “LECTIN COMPOSITIONS AND USES THEREOF,” published on Dec. 31, 1997; EP 1 002 803, having the title “Method for the isolation of legume lectins and their use as a medicament,” published on May 24, 2000; and CN 101 508 730, having the English translation of the title “Extract method for lectin of leguminous plants,” and published on Aug. 19, 2009.

The corresponding international patent publication application, namely, International Application No. PCT/PL2010/050048, filed on Aug. 12, 2010, having WIPO Publication No. WO 2012/021076 and inventor Pawel MICHALOWSKI, is hereby incorporated by reference as if set forth in its entirety herein, except for the exceptions indicated herein, for the purpose of correcting and explaining any possible misinterpretations of the English translation thereof. In addition, the published equivalents of the above corresponding international patent publication application, and other equivalents or corresponding applications, if any, in corresponding cases, and the references and documents cited in any of the documents cited herein, such as the patents, patent applications, patent publications, and other documents, except for the exceptions indicated herein, are hereby incorporated by reference as if set forth in their entirety herein except for the exceptions indicated herein.

The purpose of incorporating the corresponding foreign equivalent patent application(s), that is, PCT/PL2010/050048, is solely for the purposes of providing a basis of correction of any wording in the pages of the present application, which may have been mistranslated or misinterpreted by the translator, and to provide additional information relating to technical features of one or more embodiments, which information may not be completely disclosed in the wording in the pages of this application.

Statements made in the original foreign patent application PCT/PL2010/050048 from which this patent application claims priority which do not have to do with the correction of the translation in this patent application are not to be included in this patent application in the incorporation by reference.

Any statements about admissions of prior art in the original foreign patent application PCT/PL2010/050048 are not to be included in this patent application in the incorporation by reference, since the laws relating to prior art in non-U.S. Patent Offices and courts may be substantially different from the Patent Laws of the United States.

All of the references and documents cited in any of the patents, patent applications, patent publications, and other documents cited herein, except for the exceptions indicated herein, are hereby incorporated by reference as if set forth in their entirety herein except for the exceptions indicated herein. All of the patents, patent applications, patent publications, and other documents cited herein, referred to in the immediately preceding sentence, include all of the patents, patent applications, patent publications, and other documents cited anywhere in the present application.

Words relating to the opinions and judgments of the author of all patents, patent applications, patent publications, and other documents cited herein and not directly relating to the technical details of the description of the embodiments therein are not incorporated by reference.

The words all, always, absolutely, consistently, preferably, guarantee, particularly, constantly, ensure, necessarily, immediately, endlessly, avoid, exactly, continually, expediently, ideal, need, must, only, perpetual, precise, perfect, require, requisite, simultaneous, total, unavoidable, and unnecessary, or words substantially equivalent to the above-mentioned words in this sentence, when not used to describe technical features of one or more embodiments of the patents, patent applications, patent publications, and other documents, are not considered to be incorporated by reference herein for any of the patents, patent applications, patent publications, and other documents cited herein.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications, patent publications, and other documents cited herein may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.
   Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

What is claimed is:

1. An industrial method of producing a lectin preparation from bean seeds where the bean seeds are milled, the bean mass is extracted in an acidic environment in the presence of antioxidants and a preservative, wherein the extraction is performed at a lowered temperature, for example from 4° C. to 8° C., wherein phase mixing is performed during the initial stage of extraction and then the extract is concentrated, for example on membranes, and a thusly produced PHA fraction is supplemented with a poorly volatile preservative and is dried, for example by spraying, resulting in a lectin preparation comprising a lectin activity of no less than 128 HU/100 mg preparation.

2. A method according to claim 1, wherein the extraction is performed using an extraction system comprising acetic acid, an antioxidant, as well as a highly volatile, short-chained organic acid or its salt at a lowered temperature.

3. A method according to claim 2, wherein the solid and liquid phases are mixed in the extract during the first stage of extracting, for example within 60 minutes.

4. A method according to claim 3, wherein the second stage of the extraction process occurs without phase mixing, for example within 7 hours.

5. A method according to claim 1, wherein the solid and liquid phases are mixed in the extract during the first stage of extracting, for example within 60 minutes.

6. A method according to claim 5, wherein the second stage of the extraction process occurs without phase mixing, for example within 7 hours.

7. A method according to claim 1, wherein the second stage of the extraction process occurs without phase mixing, for example within 7 hours.

8. A method according to claim 2, wherein the second stage of the extraction process occurs without phase mixing, for example within 7 hours.

9. A feed additive for swine obtained from seeds of the common bean, wherein it comprises lectins with an activity of no less than 128 HU/100 mg of preparation with a lowered level of ballasting substances.

10. A feed additive according to claim 9, wherein it was obtained using the method according to claim 1.

11. A feed additive according to claim 9, wherein it was obtained using the method according to claim 2.

12. A feed additive according to claim 9, wherein it was obtained using the method according to claim 3.

13. A feed additive according to claim 9, wherein it was obtained using the method according to claim 4.

14. A feed additive according to claim 9, wherein it was obtained using the method according to claim 5.

15. A feed additive according to claim 9, wherein it was obtained using the method according to claim 6.

16. A feed additive according to claim 9, wherein it was obtained using the method according to claim 7.

17. A feed additive according to claim 9, wherein it was obtained using the method according to claim 8.
